SEQUENCE LISTING (31 sequences)

SEQ ID NO: 1 - protein, 15 aa, Homo sapiens
EQLQAIITASVKTES

SEQ ID NO: 2 - protein, 14 aa, Homo sapiens
VSAGARKYANQTML

SEQ ID NO: 3 - protein, 15 aa, Homo sapiens
LIPIVLHLDHGDSFE

SEQ ID NO: 4 - protein, 15 aa, Homo sapiens
YAHSRPDYVTVEGEL

SEQ ID NO: 5 - protein, 15 aa, Homo sapiens
SSSIPQEYVEMVNKY

SEQ ID NO: 6 - protein, 15 aa, Homo sapiens
DGRMVMTGTIRRLFV

SEQ ID NO: 7 - protein, 15 aa, Homo sapiens
ARKYANQTMLRYMAQ

SEQ ID NO: 8 - protein, 434 aa, Homo sapiens
MEQLQAIITASVKTESEEVSAGARKYANQTMLRYMAQEELIPIVLHLDHGDSFEEEYAHSRPDYVTVEGELEEIGTSHGAYKFPPGTKEESSSIPQEYVEMVNKYEEDGRMVMTGTIRRLFVEEAEHDAIVKECIAEAAEELRDGDKARYGGKGTQEEILNGGKHAGGNLKFQEEPFPDQQLRMVAEVYQKEELLKKHPAIVSIEDALAELDYENWTKLGLVGDDLYTTNPITIKGARGERIQKYTRLLQIYDYLKEHDMLAEEEEGRLGPSQLPWKELGIEEGYDGHLVSGAKKWLEEETKCISNASCTTNCLEEKDLRARAAGMNIIPTSTGAAIALPKVCHGLPPKSLDEETGSLVVNVNAKVEEDPIVSSDIIGCQYSSIVDALSTKVLPNPEELVKVLSWYDNEWMYSCRCADFHRLEKYLEEHHHHHH

SEQ ID NO: 9 - protein, 15 aa, Homo sapiens
AEHDAIVKECIAEAA

SEQ ID NO: 10 - protein, 15 aa, Homo sapiens
LRDGDKARYGGKGTQ

SEQ ID NO: 11 - protein, 15 aa, Homo sapiens
ILNGGKHAGGNLKFQ

SEQ ID NO: 12 - protein, 16 aa, Homo sapiens
PFPDQQLRMVAEVYQK

SEQ ID NO: 13 - protein, 15 aa, Homo sapiens
LLKKHPAIVSIEDAL

SEQ ID NO: 14 - protein, 14 aa, Homo sapiens
AELDYENWTKLGLV

SEQ ID NO: 15 - protein, 15 aa, Homo sp.
LVGDDLYTTNPITIK

SEQ ID NO: 16 - protein, 15 aa, Homo sapiens
ARGERIQKYTRLLQI

SEQ ID NO: 17 - protein, 13 aa, Homo sapiens
YDYLKEHDMLAEE

SEQ ID NO: 18 - protein, 15 aa, Homo sapiens
GRLGPSQLPWKELGI

SEQ ID NO: 19 - protein, 15 aa, Homo sapiens
GYDGHLVSGAKKVVL

SEQ ID NO: 20 - protein, 15 aa, Homo sapiens
ETKCISNASCTTNCL

SEQ ID NO: 21 - protein, 14 aa, Homo sapiens
HKDLRARAAGMNII

SEQ ID NO: 22 - protein, 15 aa, Homo sapiens
IIPTSTGAAIALPKV

SEQ ID NO: 23 - protein, 15 aa, Homo sapiens
PKVCHGLPPKSLDGG

SEQ ID NO: 24 - protein, 15 aa, Homo sapiens
TGSLVDLTVNVNAKV

SEQ ID NO: 25 - protein, 15 aa, Homo sapiens
DPIVSSDIIGCQYSS

SEQ ID NO: 26 - protein, 15 aa, Homo sapiens
DIIGCQYSSIVDALS

SEQ ID NO: 27 - protein, 15 aa, Homo sapiens
CQYSSIVDALSTKVL

SEQ ID NO: 28 - protein, 15 aa, Homo sapiens
LVKVLSWYDNEWMYS

SEQ ID NO: 29 - protein, 15 aa, Homo sapiens
SCRCADIFHRLEKYL

SEQ ID NO: 30 - protein, 434 aa, synthetic construct; identical to SEQ ID NO: 8

SEQ ID NO: 31 - protein, 316 aa, synthetic construct
EEHTYTRPEEVQDFVSKEELGEARTKLTEMYMRKEEAEHDAIVKECIAEAAEEVKYLGRVTLAARSSAEEVTLAARSSAPSGASTEETOGTVLKKNIGGNASEEDKVPKKFKLPSPFFNEEKLGGLLVKKYGLSAKEEILVKKYGLSAKNLDEFEEASSEFYDEEKKLYEVEEDYENWTKLNARLGQRVEERASRKLYPKDIQVVAEENVYLLKYDTAHRAFPEEQEFTVGEGADKWVVKEEWVVKSIGGRLGPSQLEEVVLESTGIFRTKAEKEEKDAEGKIKKDDGYDGHEETLNNAFGIRNGFMTTVHHHHHH

(19) United States
(12) Patent Application Publication
(10) Pub. No.: US 2023/0080865 A1
(43) Pub. Date: Mar. 16, 2023

(54) CHIMERIC RECOMBINANT PROTEIN ENCODING EPITOPES WITH IDENTITY TO BACTERIAL, FUNGAL, PARASITE AND HUMAN METABOLIC ENZYMES INVOLVED IN PATHOGENESIS DURING SEXUALLY TRANSMITTED INFECTIONS

(71) Applicant: Washington State University, Pullman, WA (US)

(72) Inventor: John F. ALDERETE, Moscow, ID (US)

(21) Appl. No.: 17/943,587

(22) Filed: Sep. 13, 2022

Related U.S. Application Data
(60) Provisional application No. 63/243,317, filed on Sep. 13, 2021.

(51) Int. Cl. G01N 33/569; C07K 14/44; C12N 9/88; C12N 9/02; G01N 33/564

(52) U.S. Cl. CPC G01N 33/56905; C07K 14/44; C12N 9/88; C12Y 401/02013; C12Y 402/01011; C12N 9/0008; C12Y 102/01012; G01N 33/564; G01N 2333/44; G01N 2469/20

(57) ABSTRACT

Disclosed herein are compositions and methods for detecting antibodies directed to epitopes of the metabolic enzymes, fructose-1,6-bisphosphate aldolase (A), α-enolase (E), and glyceraldehyde-3-phosphate dehydrogenase (G), that are shared between T. vaginalis and human AEG proteins.

### CROSS REFERENCE TO RELATED APPLICATIONS

This application claims priority to U.S. Provisional Patent Application No. 63/243,317 filed on Sep. 13, 2021, the disclosure of which is expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 29 kilobytes xml file named “67635-370458.xml”, created on Sep. 12, 2022.

### BACKGROUND

Sexually transmitted infections (STIs) are a major global cause of acute illness, infertility, long term disability and death, with severe medical and psychological consequences for millions of men, women and infants. WHO estimated that 340 million new cases of syphilis, gonorrhoea, chlamydia and trichomoniasis occurred throughout the world in 1999 in men and women aged 15-49 years, and incidence has risen steadily since then.

Trichomonas vaginalis causes vaginitis in women and non-gonococcal, non-chlamydial urethritis in men. For women, Trichomonas vaginalis is responsible for the number one, non-viral STI that negatively affects reproductive health. It is evident that the global prevalence of this and other STIs requires specific and sensitive point-of-care (POC) diagnostics suitable for screening large cohorts of at-risk individuals. This is especially the case at low resource settings. Among men, the most recent findings indicate a relationship between seropositivity to T. vaginalis and prostate cancer. This parasite is now the number one, non-viral sexually transmitted disease agent. In 2013, the incidence of this sexually transmitted infection (STI) referred to as trichomonosis or trichomoniasis is estimated to be 10 million women in the United States and 270 to 350 million women worldwide. Health consequences to women include cervical cancer, pelvic inflammatory disease, infertility, increased HPV and herpes susceptibility, and adverse pregnancy outcomes accompanied with low-birth-weight infants. Significantly, 25% of HIV seroconversions are the direct result of trichomonosis, which is known to increase the portal of exit and entry of HIV infectious viral particles. Therefore, control of trichomonosis may be one of the most effective means of reducing HIV transmission risk and of preventing prostate and cervical cancers worldwide.

It is clear that the public health costs as a result of this STI are enormous, and interference strategies are needed. The most important interference strategy is the availability of rapid, accurate diagnostics with exceptional sensitivity and specificity toward this STI agent. Despite the impact of this STI to public health, fundamental aspects of T. vaginalis cell biology and mechanisms of pathogenesis remain unknown. As previously disclosed in U.S. Pat. No. 8,017,103 B2, incorporated by reference herein, α-actinin is expressed by Trichomonas species and can be used to detect the presence of Trichomonas infection. However, the antibodies to parasite proteins available hitherto are inferior in their ability to detect the immunoreactive trichomonad protein antigens.

Trichomonas vaginalis is responsible for the number one, non-viral STI that negatively affects women's health. It is evident that the global prevalence of this and other STIs requires specific and sensitive point-of-care (POC) diagnostics suitable for screening large cohorts of at-risk individuals. This is especially the case at low resource settings. The POC Trichomonas Rapid Test (Sekisui Diagnostics, Lexington, Mass., USA) is useful for diagnosing Trichomonas in women, but it requires a vaginal swab from women and cannot diagnose the STI in men. There are two perfect targets that can either be used singly or in combination for a POC serodiagnostic test useful for resource-poor environments worldwide. A chimeric String-of-Epitopes (SOE) recombinant protein called AEG::SOE2 is a serodiagnostic target for this STI. This protein has been referred to as an epitope chain protein that may have applicability as a vaccine. This protein has immunogenic epitopes of fructose-1,6-bisphosphate aldolase (A), α-enolase (E), and glyceraldehyde-3-phosphate dehydrogenase (G) that are unique to the T. vaginalis proteins.

A second recombinant protein target for a serodiagnostic test, referred to as ACT::SOE3, is a 72.4-kDa truncated version of the 106.2-kDa highly immunogenic a actinin protein to which women and men patients make serum IgG antibodies. This protein is also unique to T. vaginalis in that it has no sequence identity with other proteins in databanks. This protein target has seven epitopes detected by women, within which are five epitopes reactive with men patients. Both the AEG::SOE2 and ACT::SOE3 serodiagnostics have utility for large-scale screening for this STI, which is preparatory to advancing the reproductive health of at-risk humans.

### SUMMARY

Investigation of the metabolic enzymes, fructose-1,6-bisphosphate aldolase (A), α-enolase (E), and glyceraldehyde-3-phosphate dehydrogenase (G), the “AEG” enzymes of T. vaginalis reveals the proteins are found on the surface of T. vaginalis, and provide immunogenic epitopes that are unique to T. vaginalis proteins as well as immunogenic epitopes that were not unique to T. vaginalis. In accordance with one embodiment of the present disclosure, a novel recombinant epitope chain protein is provided that comprises one or more of the non-unique epitopes of A, E and G that have ≥50% identity with homologous proteins of humans and of Candida albicans, Chlamydia trachomatis, Escherichia coli, Neisseria gonorrhoeae, Saccharomyces cerevisiae, Staphylococcus aureus, Streptococcus pneumoniae, Streptococcus pyogenes, and Treponema pallidum.

In accordance with one embodiment of the present disclosure, a recombinant polypeptide comprising a series of epitopes (SOE) linked to one another by peptide linkers is provided. In one embodiment the epitopes of the recombinant polypeptide comprise i) at least one first epitope selected from the group consisting of SEQ ID NO: 1 to SEQ ID NO: 8 which are non-unique epitopes of fructose-1,6-bisphosphate aldolase (A); ii) at least one second epitope selected from the group consisting of SEQ ID NO: 9 to SEQ ID NO: 17, which are non-unique epitopes of α-enolase (E), and iii) at least one third epitope selected from the group consisting of SEQ ID NO: 18 to SEQ ID NO: 29, which are non-unique epitopes of glyceraldehyde-3-phosphate dehydrogenase (G). In one embodiment epitopes of the SOE polypeptide are linked to one another via peptide linkers comprising 2, 3, 4, 5 or 6 amino acids, optionally wherein the amino acids of the peptide linkers are acidic amino acids, including for example glutamic acid or aspartic acid residues. In one embodiment the recombinant polypeptide comprises at least 6, 7, 8, 9, 10, 11, 12, 13, 14, 15, 16, 17, 18, 19, 20 or more epitopes selected from the amino acid sequences of SEQ ID NO: 1-29, wherein each of the epitope sequences are linked to one another via peptide bonds formed between the peptide linkers and the epitope bearing amino acid sequences of the SOE polypeptide. In one embodiment the amino acids of the peptide linkers are selected to produce a recombinant polypeptide having an isoelectric point of the SOE in a range from about 4.90 to about 5.10. In one embodiment the recombinant polypeptide comprises the sequence of SEQ ID NO: 30.

The non-unique, immunogenic epitopes of SEQ ID NOS: 1-29 have possible immuno-crossreactivity with human proteins, and thus may play a role in disease pathogenesis during T. vaginalis STI, Furthermore, infections by T. vaginalis or other pathogens that present surface AEG proteins with possible immuno-crossreactivity with human proteins. This in turn may induce the production of autoantibodies during natural infections, which may have autoimmune consequences that have heretofore been unreported and unappreciated with respect to disease outcomes resulting from host immune responses.

In accordance with one embodiment of the present disclosure, a method of detecting antibodies in a sample is provided, wherein the detected antibodies specifically bind to a polypeptide comprising one or more of the epitopes of SEQ ID NOS: 1-29. In one embodiment the method detects antibodies that specifically bind to the polypeptide of SEQ ID NO: 30. The detection of such antibodies can be used to screen for, and identify, subjects, including human patients, that are producing auto-antibodies. In one embodiment the method comprises the steps of contacting a biological sample recovered from a subject, contacting the sample with a polypeptide comprising one or more of the epitopes of SEQ ID NOS: 1-29 (optionally by contacting with the polypeptide of SEQ ID NO: 30) under conditions permissive for antigen-antibody complexes formation; and detecting formation of at least one antigen-antibody complex as an indication of the presence of at least one antibody in said sample that specifically binds to one of said series of epitopes. In one embodiment the biological sample is contacted with a polypeptide comprising the amino acid sequence of SEQ ID NO: 30 (protein AEG::SOE3).

In one embodiment the method of detecting at least one antigen-antibody complex is performed using an immunoassay, optionally an enzyme-linked immunosorbent assay (ELISA), optionally wherein the polypeptide is immobilized on a solid support. In one embodiment the method can be used to detect the presence of auto-antibodies in a biological sample of a subject.

In one embodiment a method of screening for auto-antibodies in a subject comprises obtaining a biological sample from the subject and combining the biological sample with a polypeptide including a series of epitopes (SOE), said epitopes being arranged as a linear chimeric protein array comprised of at least 8 non-unique, immunogenic epitopes of fructose-1,6-bisphosphate aldolase (A), at least 9 non-unique, immunogenic epitopes of α-enolase (E) and at least 12 non-unique, immunogenic epitopes of glyceraldehyde-3-phosphate dehydrogenase (G), with each of said epitopes being connected via a peptide bond, optionally via a peptide linker, wherein said combining is performed under conditions whereby antigen-antibody complexes are permitted to form; and detecting formation of at least one antigen-antibody complex as an indication of a presence of at least one pathogenic microorganism.

Another aspect includes a polypeptide including a series of epitopes (SOE) which includes at least 2 to 8 non-unique, immunogenic epitopes of bisphosphate aldolase (A), at least 2 to 9 non-unique, immunogenic epitopes of α-enolase (E) and at least two to 12 non-unique immunogenic epitopes of glyceraldehyde-3-phosphate dehydrogenase (G). wherein each of said epitopes is connected via a peptide bond, optionally via a peptide linker.

Another beneficial aspect includes a kit for detecting the presence of one or more pathogenic organisms, including Trichomonas vaginalis microorganisms, said kit comprising assay reagents or media for detecting formation the presence of an epitope of SEQ ID NOs: 1-29 in a biological sample.

The foregoing and other objects, features, and advantages of the present disclosure will become more apparent from the following detailed description, which proceeds with reference to the accompanying figures.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B each provide an annotate amino acid sequence of the AEG::SOE3 protein. FIG. 1A shows the linear amino acid sequence of AEG::SOE3, including the 436 amino acid SOE, with the hexa-His tag at the carboxy terminal end. The peptide-epitope sequences are linked by EE (Glutamic Acid) amino acids (underlined). Linear sequence with A, E and G refer to amino acid sequences from the T. vaginalis proteins, respectively, as described for Table 1. The epitopes were detected by pooled positive women (W) or men (M) sera, as shown in FIG. 1B, and the number next to W and M represents the order the epitope was identified within the protein during epitope mapping on the SPOTs system. The monoclonal antibodies (MAbs) labeled ALD 11, ALD 12, ALD 13, ALD 25, B44 and B43 are reactive with the epitopes, as indicated.

FIG. 2 shows the representative SDS-PAGE in 8% acrylamide of the 35.9-kDa AEG::SOE2 protein (lane 2) and the 49.4-kDa AEG::SOE3 protein (lane 3). Both proteins were purified by Ni2+NTA affinity chromatography. Lane 1 is of molecular weight (MW) standards, and numbers refer to daltons (kDa, ×1000).

FIG. 3 shows the representative ELISA for detection of 1 μg AEG::SOE3 immobilized onto individual wells of 96-well microtiter plates. The negative control is the secondary peroxidase-conjugated goat anti-mouse IgG. Normal mouse serum (NMS), mouse anti-AEG::SOE2 serum and mouse anti-T. vaginalis (Tv) serum were each diluted 1:10 in eBSA-PBS. The AEG::SOE3 MAb cocktails 1, 2 and 3 were as follows: mix1 included ALD12, ALD11, ALD25, mix 2 included ALD12, B44, B43 and mix 3 included ALD12, ALD1, ALD25, B44, B43 (FIG. 1B), and cocktails included equal volumes of each MAb. The AEG::SOE2 MAb cocktail of MAbs ALD13, ALD55, ALD30 and ALD32, and the mouse anti-AEG::SOE2 serum. Both MAbs and antiserum were unreactive with AEG::SOE3. Mouse anti-T. vaginalis (Tv) serum was generated to T. vaginalis isolate NYH 286. The cocktails of MAbs and the individual MAbs ALD11 and ALD12 detected AEG::SOE3. The use of pooled MAbs or individual MAbs are as described herein. As expected, negative control irrelevant MAbs HA423 to α-actinin and L64 to a cytoplasmic protein of T. vaginalis were unreactive to AEG::SOE3. The ELISA was repeated on four different times with similar results.

### DETAILED DESCRIPTION

### Definitions

In describing and claiming the invention, the following terminology will be used in accordance with the definitions set forth below.

The following are definitions of terms that may be used in the present specification. The initial definition provided for a group or term herein applies to that group or term throughout the present specification individually or as part of another group, unless otherwise indicated.

Additionally, it will be understood that any list of such candidates or alternatives is merely illustrative, not limiting, unless implicitly or explicitly understood or stated otherwise.

As used in this specification and the appended claims, the singular forms “a”, “an” and “the” include plural referents unless the content clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

In addition, unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

As used herein the term “patient” or “subject” without further designation is intended to encompass any warm blooded vertebrate domesticated animal (including for example, but not limited to livestock, horses, cats, dogs and other pets) and humans receiving therapeutic care with or without physician oversight.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein the term “solid support” relates to a solvent insoluble substrate that is capable of forming linkages (preferably covalent bonds) with soluble molecules. The support can be either biological in nature, such as, without limitation, a cell or bacteriophage particle, or synthetic, such as, without limitation, an acrylamide derivative, glass, plastic, agarose, cellulose, nylon, silica, or magnetized particles. The support can be in particulate form or a monolythic strip or sheet. The surface of such supports may be solid or porous and of any convenient shape.

The term “linked” or like terms refers to the connection between two groups. The linkage may comprise a covalent, ionic, or hydrogen bond or other interaction that binds two compounds or substances to one another.

As used herein the term “acidic amino acid” refers to an amino acid that comprises a second acidic moiety (other than the alpha carboxylic acid of the amino acid), including for example, a side chain carboxylic acid or sulfonic acid group.

### EMBODIMENTS

One aspect of the present disclosure is directed to polypeptides that comprises a series of non-unique, immunogenic epitopes (SEQ ID NOS: 1-29) associated with the metabolic enzymes, fructose-1,6-bisphosphate aldolase (A), α-enolase (E), and glyceraldehyde-3-phosphate dehydrogenase (G), the “A”, “E” and “G” enzymes of T. vaginalis. Antibodies raised against these epitopes are anticipated to cross-react with native human antigens and therefore in one embodiment the polypeptides comprising a series of one or more non-unique, immunogenic epitopes of SEQ ID NOS: 1-29 may be used to detect autoantibodies in a patient's biological fluids.

In accordance with one embodiment, the disclosed technology is generally directed to a construct of a 49.41-kDa AEG::SOE3 chimeric protein of pl 4.83 comprised of 8, 9 and 12 non-unique, immunogenic epitopes of A, E and G, respectively. These non-unique epitopes were detected by sera of women and men patients but not control, seronegative sera of women and men. The epitopes were found to have ≥50% up to 100% sequence amino acid sequence identity with the human homolog and homologs of Candida albicans, Escherichia coli, Saccharomyces cerevisiae, Staphylococcus aureus, Streptococcus pneumoniae, Streptococcus pyogenes as well as the homologs of the STI agents Chlamydia trachomatis, Neisseria gonorrhoeae and Treponema pallidum. The AEG::SOE3 protein was not detected by mouse anti-AEG::SOE2 serum and by monoclonal antibodies (MAbs) to AEG::SOE2, showing the distinctness of these two chimeric proteins (see U.S. Pat. No. 10,677,798, the disclosure of which is expressly incorporated herein, for details regarding SOE2). AEG::SOE3 was detected by ELISA and immunoblot with sera of mice immunized with fixed T. vaginalis organisms, with sera of women and men patients with trichomonosis, and with MAbs to AEG::SOE3. The patient sera reactive to these immunogenic metabolic enzyme epitopes of AEG::SOE3 may be indicative of immune-crossreactive antibodies to human proteins during this STI. The role of these possible immune-crossreactive antibodies produced during this STI and those conceivably generated by other STIs and microbial pathogens to heretofore unappreciated disease pathogenesis is discussed.

It is also to be noted that applicable contents and methods of U.S. Pat. No. 9,910,042, entitled: “Strings of epitopes useful in diagnosing and eliciting immune responses to sexually transmitted infections,” U.S. Pat. No. 10,386,369, entitled: “Strings of epitopes useful in diagnosing and eliciting immune responses to sexually transmitted infections,” and 10,677,798, entitled: “Strings of epitopes useful in diagnosing and eliciting immune responses to sexually transmitted infections,” all to John, F. Alderete, the inventor of the present application, are incorporated herein by reference.

In accordance with one embodiment of the present invention a recombinant polypeptide is provided comprising a string of epitope bearing peptide fragments wherein the peptide fragments are linked to one other via peptide linkers and the recombinant polypeptide comprises at least six, nine, twelve, fifteen or eighteen epitope bearing peptide fragments selected from the group consisting of SEQ ID NOS: 1-29. In one embodiment the recombinant polypeptide comprises 2, 3, 4, 5, 6, 7, 8, 9, 10, 11, 12, 13, 14, 15, 16, 17, 18, 19, 20, 21, 22, 23, 24, 25, 26, 27, 28 or 29 epitope bearing peptide fragments selected from the group consisting of SEQ ID NOS: 1-29. In one embodiment a single epitope bearing peptide fragment may comprising two or more epitope sequences selected from SEQ ID NOS: 1-29. In one embodiment the recombinant polypeptide comprises each of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29, wherein each of said epitopes are linked to one another via a plurality of peptide linkers, in a linear array. In one embodiment the SOE polypeptide comprises the sequence of SEQ ID NO: 30.

The SOE polypeptides of the present disclosure comprising a plurality of epitope bearing peptide fragments selected from the group consisting of SEQ ID NOS: 1-29, wherein the epitope bearing peptide fragments are linked to one another via a plurality of peptide linkers that join the carboxy terminus of the preceding epitope bearing peptide fragment to the amino terminus of the next epitope bearing peptide fragment of the SOE polypeptide. The peptide linker can be any length, but typically is less than 10 amino acids, and more typically is 6, 5, 4, 3 or 2 amino acids in length. The plurality of peptide linkers present in the SOE polypeptide can individually be of different lengths, but in one embodiment each of the peptide linkers of the SOE polypeptide are the same length, and optionally the same amino acid content.

The amino acids comprising the peptide linker can be selected from any of the known or standard amino acids. In one embodiment the amino acids of the peptide linker are selected to optimize the isoelectric point of the SOE polypeptide to a range from about 4.90 to about 5.10. In one embodiment the amino acids of the peptide linker consist of polar amino acids. In one embodiment the amino acids of the peptide linker consist of acidic amino acids, and in one embodiment amino acids of the peptide linker are selected from the group consisting of Glu, Asp, homoglutamic acid and homocysteic acid. In one embodiment the peptide linker is 2 to 4 amino acids in length wherein the amino acids are selected from the group consisting of Glu, Asp, homoglutamic acid and homocysteic acid. In some embodiments, the peptide linkers are repeats of amino acid residues such as glycine (-GG-), lysine (—KK—), glutamic acid (-EE-), and mixtures thereof within the SOE. In one embodiment the peptide linker is 2 amino acids in length wherein the amino acids are selected from the group consisting of Glu and Asp. In one embodiment the peptide linker is Glu-Glu or Asp-Asp. In one embodiment the SOE polypeptide comprises a mixture of two or more of -GG-, —KK—, and -EE- peptide linkers.

In accordance with one embodiment the SOE polypeptides of the present disclosure are immobilized on a solid support. The solid support may be in particulate form or may be a monolith. In one embodiment the solid support is part of a lateral flow or vertical flow through device. In one embodiment the solid support comprises a homogenous population of SOE polypeptides. In one embodiment the solid support comprises a heterogeneous population of SOE polypeptides wherein the SOE polypeptides differ in length and/or epitope content. In one embodiment the SOE polypeptides are localized to a specific region of the solid support. In one embodiment the solid support comprises the SOE polypeptide of SEQ ID NO: 30. In one embodiment the solid support comprises a first region comprising AEG::SOE2 (SEQ ID NO: 31) and a second region comprising AEG::SOE3 (SEQ ID NO: 30).

In accordance with one embodiment a method is provided for detecting antibodies that bind to a recombinant polypeptide comprises two or more epitope bearing peptide fragments selected from the group consisting of SEQ ID NOS: 1-29. In one embodiment the method comprises screening a biological sample obtained from a patient for the presence of such antibodies. In one embodiment the method comprises the steps of contacting the sample with an SOE polypeptide of the present disclosure under conditions permissive for antigen-antibody complexes formation; and detecting formation of at least one antigen-antibody complex as an indication of a presence of at least one antibody in said sample that specifically binds to the SOE polypeptide. In one embodiment the detection of antigen-antibody complexes is performed using an immunoassay, including for example an enzyme-linked immunosorbent assay (ELISA).

The sample used in the antibody detection method of the present disclosure can be any biological material where antibodies may be detected. In one embodiment the biological sample is selected from the group consisting of serum, plasma, blood, saliva, semen, cerebrospinal fluid, semen, prostatic fluid, urine, sputum, joint fluid, body cavity fluid, whole cells, cell extracts, tissue, biopsy material, aspirates, exudates, vaginal washings, pap smear samples, pap smear preparations, slide preparations, fixed cells, and tissue sections. In one embodiment the sample is selected from the group consisting of serum, plasma, blood, saliva, semen, urine, and sputum, and in one embodiment the sample is serum or plasma. In one embodiment the biological sample is recovered from a subject selected from the group consisting of human, non-human primate, dog, cat, cattle, sheep, swine, horse, bird, mouse, and rat. In one embodiment the sample is recovered from a human.

The detection of such antibodies can be used as an indicator of a pathogenic infection or as an indicator of the presence of auto-antibodies. In accordance with one embodiment a method of treating a patient with an autoimmune disease is provided wherein the patient is screened for the presence of antibodies that bind to an SOE polypeptide comprising two or more epitopes selected from SEQ ID NOS: 1-18, optionally the SOE3 polypeptide of SEQ ID NO: 30, wherein patient's whose sample detected antibodies specifically binding to the SOE polypeptide are then treated with immunomodulating agents.

It should be emphasized that the above-described embodiments and following specific examples of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

### Example 1

Non-Unique Epitopes to the A, E and G Proteins of T. vaginalis.

The SPOTs system (Custom Peptide Array; Sigma-Aldrich Corp, St Louis, Mo., USA) is currently used for determining the epitopes of fructose-1,6-bisphosphate aldolase (A), α-enolase (E), and glyceraldehyde-3-phosphate dehydrogenase (G). There are specific protocols used for deriving the oligopeptides from probing the SPOTs membranes with women and men positive control sera by using the GenBank accession numbers for each protein. Sequence identity analysis with the enzyme homologs of human and other eukaryote and bacterial organisms has been reported. The Immune Epitope Database and Analysis Resource was used to show the linear nature of the epitope sequences.

### Plasmid Encoding AEG::SOE3

GenWay Biotech, Inc (San Diego, Calif.) synthesized the DNA coding sequence for AEG::SOE3 (SEQ ID NO: 30) in the pET-23a(+) plasmid with ampicillin and chloramphenicol genes. The protein was synthesized by induced E. coli BL21DE3 cells. AEG::SOE3 has hexa-histidine at the carboxy terminus for purification.

### Recombinant AEG::SOE3

The E. coli was grown on Luria Broth with the antibiotics. Briefly, E. coli were grown at 37° C. to OD600. The flask of E. coli was then transferred to shaking incubator at 18° C. prior to induction with isopropyl-β-D-thiogalactopyranoside and incubated for 16-hours. Ni²⁺-NTA superflow affinity column chromatography (Qiagen Inc., Valencia, Calif., USA) was used to purify the protein.

### Enzyme-Linked Immunosorbent Assay (ELISA) for Detection of Proteins.

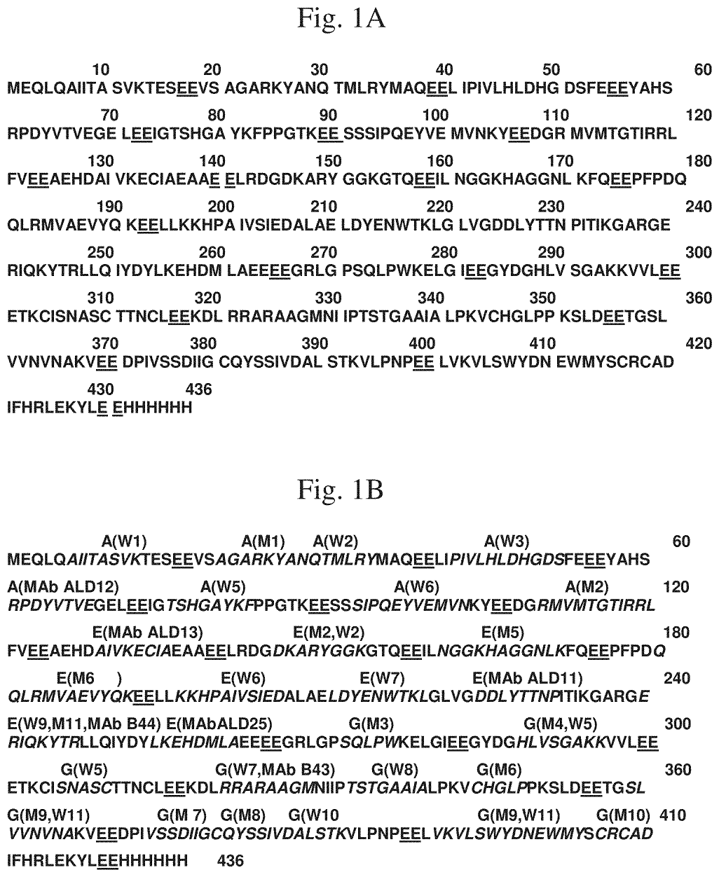

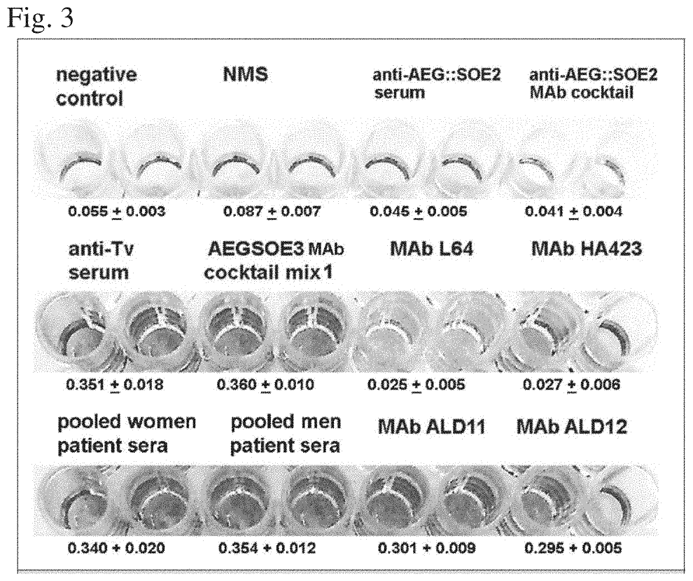

Sera from women and men was used for detection of proteins by ELISA. Institutional Review Board (IRB) approvals for collecting and using the sera were obtained. Reactivities with AEG::SOE2 were compared with this new AEG::SOE3 protein (Table 2) using well-established protocols. The microtiter plates were prepared for ELISA. Monoclonal antibodies (MAbs) to AEG::SOE3 are shown in FIG. 1B. When using MAbs singly for experiments, a 50 μl volume of undiluted hybridoma supernatant was added to wells. Alternatively, a cocktail of 25 μl for each of 4 MAbs (FIG. 3 and Table 2) were used. A 2% ELISA-grade bovine serum albumin (eBSA) (Sigma Chemical Co., St. Louis, Mo.) prepared in PBS (eBSA-PBS) was used for blocking the microtiter plates and for dilutions of sera.

Comparing Mouse Anti-AEG::SOE2 Serum and Mouse Anti-T. vaginalis Serum and ELISA for Detecting Antibody to AEG::SOE3.

Mouse anti-AEG::SOE2 serum was generated. Anti-T. vaginalis serum generated in mice immunized with glutaraldehyde-fixed trichomonads was used.

### Reproducibility

Experiments were performed at least three times under identical conditions. ELISAs on microtiter plates were always in quadruplicate. Averages and standard deviations were calculated.

The T. vaginalis Non-Unique, Immunogenic Epitopes of a, E, and G and the AEG::SOE3 Protein

Epitope mapping revealed that pooled women and men positive serum recognized 12 epitopes of fructose-1,6-bisphosphate aldolase (A), 18 epitopes of α-enolase (E), and 19 epitopes of glyceraldehyde-3-phosphate dehydrogenase (G) for a total of 49 epitopes for the three proteins. FIG. 1B shows there were only 8 of 12, 9 of 18, and 12 of 19 non-unique, immunogenic epitopes for a total of 29 epitopes to the T. vaginalis A, E, and G proteins, respectively (See Table 1). These epitope amino acid sequences had ≥50% sequence identity with other bacterial, fungal and human sequences. The amino acid sequences are the epitopes identified by IgG antibody in the SPOTS system. The first sequence for E had no identity with other proteins in databanks and is included as an internal control because the epitope is recognized by monoclonal antibody (MAb) ALD 13 (FIG. 1B).

TABLE 1

| | | glyceraldehyde- |
|---|---|---|
| fructose-1,6- | | 3-phosphate |
| bisphosphate | | dehydrogenase |
| aldolase (A) | α-enolase (E) | (G) |
| EQLQAIITASVKTES | AEHDAIVKECIAEAA | GRLGPSQLPWKELGI |
| (SEQ ID NO: 1) | (SEQ ID NO: 9) | (SEQ ID NO: 18) |
| VSAGARKYANQTML | LRDGDKARYGGKGTQ | GYDGHLVSGAKKVVL |
| (SEQ ID NO: 2) | (SEQ ID NO: 10) | (SEQ ID NO: 19) |
| ARKYANQTMLRYMAQ | ILNGGKHAGGNLKFQ | ETKCISNASCTTNCL |
| (SEQ ID NO: 7) | (SEQ ID NO: 11) | (SEQ ID NO: 20) |
| LIPIVLHLDHGDSFE | PFPDQLRMVAEVYQK | HKDLRRARAAGMNII |
| (SEQ ID NO: 3) | (SEQ ID NO: 12) | (SEQ ID NO: 21) |
| YAHSRPDYVTVEGEL | LLKKHPAIVSIEDAL | IIPTSTGAAIALPKV |
| (SEQ ID NO: 4) | (SEQ ID NO: 13) | (SEQ ID NO: 22) |
| IGTSHGAYKFPPGTK | AELDYENWTKLGLV | PKVCHGLPPKSLDGG |
| (SEQ ID NO: 8) | (SEQ ID NO: 14) | (SEQ ID NO: 23) |
| SSSIPQEYVEMVNKY | LVGDDLYTTNPITIK | TGSLVDLTVNVNAKV |
| (SEQ ID NO: 5) | (SEQ ID NO: 15) | (SEQ ID NO: 24) |
| DGRMVMTGTIRRLFV | ARGERIQKYTRLLQI | DPIVSSDIIGCQYSS |
| (SEQ ID NO: 6) | (SEQ ID NO: 16) | (SEQ ID NO: 25) |
| | YDYLKEHDMLAEE | DIIGCQYSSIVDALS |
| | (SEQ ID NO: 17) | (SEQ ID NO: 26) |
| | | CQYSSIVDALSTKVL |
| | | (SEQ ID NO: 27) |
| | | LVKVLSWYDNEWMYS |
| | | (SEQ ID NO: 28) |
| | | SCRCADIFHRLEKYL |
| | | (SEQ ID NO: 29) |

### Purification of Recombinant AEG::SOE2

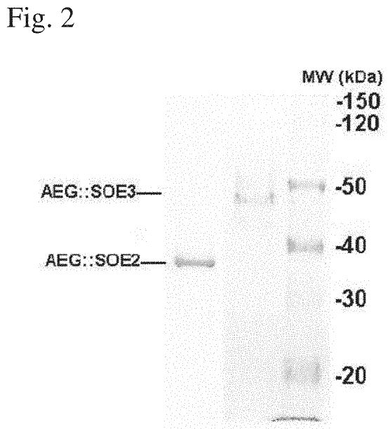

FIG. 2 shows the Coomassie Brilliant blue-stained gel after sodium dodecyl sulfate-polyacrylamide gel electrophoresis (SDS-PAGE) of 2 μg purified AEG::SOE3 (lane 3) and is compared with 1 μg of the AEGSOE2 protein (lane 2). The relative mobility of the proteins were compared with molecular weight standards (MW, lane 1) in kilodaltons (kDa) and are consistent with the expected size of ˜36-kDa for AEGSOE2 and ˜49.4-kDa for AEG::SOE3.

ELISA Using AEG::SOE3-Coated Wells Probed with Different Antibodies.

ELISA was then performed with 1 μg AEG::SOE3 immobilized onto microtiter wells, and representative results are shown in FIG. 3. The protein was detected by mouse anti-T. vaginalis (Tv) serum diluted 1:10, three different cocktails of MAbs and the individual MAbs ALD 11 and ALD12. Not surprisingly, the AEG::SOE3 recombinant protein (FIG. 2) was detected by immunoblot with these antibody reagents (not shown). Not unexpectedly, there was no detection of AEG::SOE3 by various negative controls, including the secondary peroxidase-conjugated goat anti-mouse IgG (negative control), which gave results equal to normal mouse serum (NMS). Both the mouse anti-AEG::SOE2 serum and a cocktail of MAbs to AEG::SOE2 were unreactive with AEG::SOE3. Two irrelevant MAbs of the same isotype to the α-actinin protein (MAb HA423) and to a cytoplasmic protein (MAb L64) of T. vaginalis were unreactive to AEG::SOE3.

Representative ELISA Comparing ACT-P2 and AEG::SOE2 with Different Antibodies.

AEG::SOE3 was compared with AEG::SOE2 for detection of IgG with different antibodies. Table 2 presents results from a representative experiment that was repeated at least four different times. The negative controls included the ELISA-grade BSA in PBS (eBSA-PBS) alone, secondary peroxidase-conjugated goat anti-mouse IgG, normal mouse serum, and the MAbs L64 and HA423. As expected, the mouse anti-AEG::SOE2 serum was reactive with AEG::SOE2 but not AEG::SOE3. The mouse anti-T. vaginalis (Tv) serum readily detected both constructs. Not surprisingly, three different cocktails of MAbs (FIG. 1B) detected AEG::SOE3 but not AEG::SOE2. Similarly, MAbs to AEG::SOE2 were unreactive with AEG::SOE3.

TABLE 2
Representative ELISA comparing sera and monoclonal antibodies
(MAbs) for detection of AEG::SOE3 and AEG::SOE2 as targets.

| sera and MAbs^{‡} | dilution | AEG::SOE3 | AEG::SOE2 |
|---|---|---|---|
| eBSA-PBS^{‡‡} | none | 0.067 ± 0.007^{‡} | 0.052 ± 0.005 |
| secondary | 1:1000 | 0.111 ± 0.020 | 0.110 ± 0.023 |
| peroxidase-goat | | | |
| anti-mouse IgG | | | |
| normal mouse | 1:10 | 0.116 ± 0.010 | 0.113 ± 0.0115 |
| serum | | | |
| mouse anti- | 1:10 | 0.104 ± 0.020 | 0.318 ± 0.015 |
| AEG::SOE2 serum | | | |
| mouse anti-Tv | 1:10 | 0.295 ± 0.022 | 0.318 ± 0.012 |
| serum | | | |
| AEG::SOE3 MAb | 1:1:1 | 0.301 ± 0/020 | 0.105 ± 0.010 |
| cocktail mix 1 | (v/v) | | |
| AEG::SOE3 MAb | 1:1:1 | 0.312 ± 0.015 | 0.110 ± 0.015 |
| cocktail mix 2 | (v/v) | | |
| AEG::SOE3 MAb | 1:1:1 | 0.308 ± 0.023 | 0.100 ± 0.021 |
| cocktail mix 3 | (v/v) | | |
| AEG::SOE2 MAb | 1:1:1:1 | 0.104 ± 0.018 | 0.341 ± 0.010 |
| cocktail | (v/v) | | |
| MAb L64 negative | none | 0.050, 0.055 | 0.060, 0.055 |
| control | | | |
| HA423 negative | none | 0.055, 0.055 | 0.051, 0.050 |
| control | | | |

AEG::SOE3 is distinguished from AEG::SOE2 by the fact that the epitopes of fructose-1,6-bisphosphate aldolase (A), α-enolase (E), and glyceraldehyde-3-phosphate dehydrogenase (G) are not unique to T. vaginalis and share ≥50% identity with the human homologue as well as homologues of C. albicans, C. trachomatis, E. coli, N. gonorrhoeae, S. cerevisiae, S. aureus, S. pneumoniae, S. pyogenes and T. pallidum. The positive sera of women and men patients with T. vaginalis reacted with AEG::SOE3. Importantly, the mouse anti-AEG::SOE2 sera comprised of different A, E, and G epitopes that were unique to T. vaginalis was unreactive with AEG::SOE3 (Table 2). This fact shows the specific and distinct nature of the different epitopes and the two proteins. Not surprisingly, the MAbs to respective proteins only reacted uniquely to each protein (Table 2). The observation that the anti-T. vaginalis serum of mice immunized with chemically-stabilized organisms detected AEG::SOE3 and AEG::SOE2 reaffirms that the enzymes reside on the surface of T. vaginalis, to which the host immune response makes IgG antibodies to specific epitopes.

It was surprising and unexpected that metabolic enzymes like A, E and G would be highly immunogenic proteins that elicit serum antibody IgG responses. Furthermore, it may not have been predictable that the host immune response would make IgG antibodies to 12 immunogenic epitopes of A, 18 epitopes of E and 19 epitopes of G, especially as these enzymes are highly conserved among humans and microorganisms. Equally surprising and unexpected was that 8 of 12 epitopes of A, 9 of 18 epitopes of E and 12 of 19 epitopes of G, for a total of 29 epitopes, are non-unique and have ≥50% sequence identity with homologues of humans and bacterial pathogens and fungi. This fact may suggest that assumptions that the immune response to microbial pathogens may not aggressively be towards protein homologues of humans requires additional and/or renewed attention.

The E and G proteins are on the surface of T. vaginalis, and aldolase is a surface-associated protein. Surface-associated proteins are ligands for binding host pathogenicity proteins, like plasminogen and other proteins, such as fibronectin, laminin, and collagen. It is conceivable that an antibody response to these auto-epitopes is indicative of the infected host's immune recognition of these proteins not as homologs of humans but as virulence factors involved in disease pathogenesis.

It is important to consider that antibodies to these non-unique trichomonad enzyme epitopes may elicit auto-immune reactions. Possible tissue damage may result from this STI (see Table 1) as a result of these autoantibodies. This is an issue that should be considered within the framework of pathogenesis of trichomonosis. Human serum antibody to α-enolase of group A streptococcus cross-reacts with host tissues. Although it is not known whether bacteria, viruses, parasites and fungi protein epitopes have identity to human proteins, it is a fact that these organisms are now considered triggers of autoimmunity. For example, hepatitis C in patients is considered in cases of autoimmune encephalitis due to West Nile virus and other bacteria. The role of viruses and microbial pathogens is related to multiple sclerosis. Among some organisms implicated in diseases are the following: Chlamydia pneumoniae and biliary cirrhosis, Helicobacter pylori and autoimmune gastritis, Trypanosoma cruzi and Chagas' cardiomyopathy, Leishmania and autoimmune hepatitis and Mycoplasma arthritidis and rheumatoid arthritis. Therefore, the epitopes presented in Table 1 with sequence identities to human and other microbial pathogen proteins may play a role in heretofore unknown disease pathogenesis via eliciting autoantibodies crossreactive with host proteins.

The existence of immuno-cross reactive protein epitopes mentioned above is a concern with using whole cell or lysates as diagnostics and/or vaccines. It is important that a serodiagnostic test target be comprised of a protein and/or epitopes unique only to the pathogen of interest. This is because a target with non-unique, immunogenic epitopes may lead to false positive diagnosis for the microbial pathogen of interest. Likewise, a pathogen with crossreactive epitopes to human proteins is unsuitable as a vaccine and may contribute to adverse autoimmune reactions. The examination of AEG::SOE3 as the target for IgG found in convalescent sera of patients who were infected with any of the other microbial pathogens mentioned above may be important. A positive result would be a significant finding. This is due to the nature of the extent of crossreactivity to AEG::SOE3 among infectious diseases, which could mean we would reconsider the actual mediators of disease pathogenesis.

In this context, a new paradigm for an IgG antibody to metabolic enzymes during immune responses to infectious diseases is disclosed herein. AEG::SOE3 with non-unique immunogenic epitopes of T. vaginalis A, E and G has merit as the diagnostic. On the one hand, a positive result for antibody to epitopes with sequence identity to proteins homologous with human and other organisms would not inform as to the pathogen(s) involved in the disease. On the other hand, when considering that trichomonosis is a significant asymptomatic disease and that women and men may be infected for long periods of time, such a positive serum IgG test result indicates the presence of autoantibodies, which may have consequences heretofore not considered for this STI. Because autoantibodies are made during this STI and the fact that epitope sequences have identity to other STI organisms and other infectious agents, one must now consider sequelae that may have been ignored previously.

The epitope chain protein AEG::SOE3 comprised of non-unique, immunogenic epitopes of A, E and G of Trichomonas vaginalis is the main embodiment. The IgG antibody of women and men patients is made to epitopes with sequence identity to human enzymes and that may be crossreactive to epitopes common to other microorganism and fungi. Other infectious diseases, such as those indicated in this specification, may also be producing autoantibodies, and the existence of immuno-crossreactive antibodies to human enzymes may have consequences that have neither previously been considered nor appreciated for mechanisms of disease pathogenesis.

1. A recombinant polypeptide comprising a series of epitopes (SOE) wherein said epitopes comprise
i) at least one first epitope selected from the group consisting of SEQ ID NO: 1 to SEQ ID NO: 8;
ii) at least one second epitope selected from the group consisting of SEQ ID NO: 9 to SEQ ID NO: 17; and
iii) at least one third epitope selected from the group consisting of SEQ ID NO: 18 to SEQ ID NO: 29, wherein each of said epitopes are linked to one another via a plurality of peptide linkers, in a linear array.

2. The recombinant polypeptide of claim 1 wherein the peptide linkers joining the first, second and third epitopes are 2 to 5 amino acids in length.

3. The recombinant polypeptide of claim 2 wherein the peptide linkers are comprised of acidic amino acids.

4. The recombinant polypeptide of claim 1 wherein each of the plurality of peptide linkers joining the epitopes are identical.

5. The recombinant polypeptide of claim 4 wherein the peptide linkers consist of a dipeptide of glutamic acid.

6. The recombinant polypeptide of claim 4 wherein the SOE includes at least six epitopes.

7. The recombinant polypeptide of claim 4 wherein the SOE includes at least nine epitopes.

8. The recombinant polypeptide of claim 4 wherein the SOE includes at least twelve epitopes.

9. The recombinant polypeptide of claim 1, wherein the isoelectric point of the SOE is in a range from about 4.90 to about 5.10.

10. The recombinant polypeptide of claim 1 wherein the polypeptide comprises the sequence of SEQ ID NO: 30.

11. The recombinant polypeptide of claim 1 wherein the polypeptide is linked to a solid support.

12. A method of detecting antibodies in a sample that specifically bind to the polypeptide of claim 1, said method comprising the steps of
contacting said sample with the polypeptide of claim 1 under conditions permissive for antigen-antibody complexes formation; and
detecting formation of at least one antigen-antibody complex as an indication of a presence of at least one antibody in said sample that specifically binds to one of said series of epitopes.

13. The method of claim 12, wherein said detecting step is performed using an immunoassay.

14. The method of claim 13, wherein said immunoassay is an enzyme-linked immunosorbent assay (ELISA).

15. The method of claim 12 wherein the sample is from a subject being screened for the presence of auto-antibodies.

16. The method of claim 15, wherein said sample is selected from the group consisting of serum, plasma, blood, saliva, semen, urine, and sputum.

17. The method of claim 16, wherein said subject is a human.

18. The method of claim 12, wherein the sample is contacted with a composition comprising a plurality of polypeptides of claim 1, wherein the plurality of polypeptides do not share the same amino acid sequence.

19. The method of claim 12, wherein the sample is contacted with a composition comprising a polypeptide of SEQ ID NO: 30.

20. The method of claim 19, wherein the polypeptide is linked to a solid support.
